(19) United States
(12) Patent Application Publication
(10) Pub. No.: US 2005/0149057 A1
(43) Pub. Date: Jul. 7, 2005

(54) IOL inserter plunger and body interface

(76) Inventor: Brian D. Rathert, St. Petersburg, FL (US)

Correspondence Address: Bausch & Lomb Incorporated, One Bausch & Lomb Place, Rochester, NY, 14604-2701, US

(21) Appl. No.: 10/744,981

(22) Filed: Dec. 22, 2003

(51) Int. Cl. A61F 9/00

(52) U.S. Cl. 606/107

(57) ABSTRACT

An injector body and plunger for injecting an IOL into an eye includes one or more cantilevered projections attached to the plunger shaft to provide a resistive force between the plunger and injector body. Detents may be strategically positioned along the injector body for engaging with the free ends of the projections to selectively locate the longitudinal position of the plunger relative to the body.

### BACKGROUND OF THE INVENTION

The present invention relates to ophthalmic surgical devices. More particularly, the present invention relates to a device for inserting an intraocular lens (IOL) into an eye.

IOLs are artificial lenses used to replace the natural crystalline lens of the eye when the natural lens has cataracts or is otherwise diseased. IOLs are also sometimes implanted into an eye to correct refractive errors of the eye in which case the natural lens may remain in the eye together with the implanted IOL. The IOL may be placed in either the posterior chamber or anterior chamber of the eye. IOLs come in a variety of configurations and materials. Some common IOL styles include the so-called open-looped haptics which include the three-piece type having an optic and two haptics attached to and extending from the optic; the one-piece type wherein the optic and haptics are integrally formed (e.g., by machining the optic and haptics together from a single block of material); and also the closed looped haptic IOLs. Yet a further style of IOL is called the plate haptic type wherein the haptics are configured as a flat plate extending from opposite sides of the optic. The IOL may be made from a variety of materials or combination of materials such as PMMA, silicone, hydrogels and silicone hydrogels, etc.

Various instruments and methods for implanting the IOL in the eye are known. In one method, the surgeon simply uses surgical forceps having opposing blades used to grasp the IOL and insert it through the incision into the eye. While this method is still practiced today, more and more surgeons are using more sophisticated IOL inserter devices which offer advantages such as affording the surgeon more control when inserting the IOL into the eye. IOL inserter devices have recently been developed with reduced diameter insertion tips which allow for a much smaller incision to be made in the cornea than is possible using forceps alone. Smaller incision sizes (e.g., less than about 3 mm) are preferred over larger incisions (e.g., about 3.2 to 5+ mm) since smaller incisions have been attributed to reduced post-surgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL inserter device must therefore be designed in such a way as to permit the easy passage of the IOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Thus, as explained above, the IOL inserter device must be designed to permit easy passage of the IOL therethrough. It is equally important that the IOL be expelled from the tip of the IOL inserter device and into the eye in a predictable orientation and manner. Should the IOL be expelled from the tip too quickly or in the wrong orientation, the surgeon must further manipulate the IOL in the eye possibly resulting in trauma to the surrounding tissues of the eye. It is therefore highly desirable to have an inserter device which allows for precise loading of the IOL into the inserter device and which will pass and expel the IOL from the inserter device tip and into the eye in a controlled, predictable and repeatable manner.

To ensure controlled expression of the IOL through the tip of the IOL inserter device, the IOL must first be loaded into the IOL inserter device. The loading of the IOL into the inserter device is therefore a precise and very important step in the process. Incorrect loading of an IOL into the inserter device is oftentimes cited as the reason for a failed IOL delivery sequence. Many IOL inserter devices on the market today require the IOL to be loaded into the inserter at the time of surgery by the attending nurse and/or surgeon. Due to the delicate nature of the IOL, there is a risk that the nurse and/or surgeon will inadvertently damage the IOL and/or incorrectly load the IOL into the inserter device resulting in a failed implantation.

In a typical IOL inserter device, the IOL inserter utilizes a plunger having a tip which engages the IOL (which has been previously loaded and compressed into the inserter lumen) to pass it through the inserter lumen. The IOL thus interfaces with the plunger tip as well as the lumen of the inserter device. The lumen typically is dimensioned with a narrowing toward the open tip thereof in order to further compress the IOL as it is advanced through the lumen. The tip of the lumen is sized for insertion through the surgical incision which, as stated above, is presently preferred in the sub 3 mm range. Thus, an inserter lumen will typically be dimensioned larger at the load area of the IOL and gradually decrease in diameter to the tip of the lumen where the IOL is expressed into the eye. It will be appreciated that the compressed diameter of the IOL at the lumen tip is the same as the inner diameter of the lumen tip, preferably sub 3 mm as stated above. Each of these component interfaces are dynamic in the sense that the forces acting between the interfacing components (i.e., the IOL, the plunger tip and the inserter lumen) will vary as the IOL is pushed through the lumen. Control of these dynamic forces is therefore of utmost importance or otherwise the IOL may be damaged during delivery due to excessive compressive forces acting thereon. For example, as the IOL is advanced by the plunger through an ever-decreasing diameter lumen, the IOL is being compressed while at the same time the forces necessary to push the IOL through the lumen increase. This may lead to excessive force between the plunger tip and the IOL resulting in possible damage to the IOL and/or uncontrolled release of the IOL from the lumen tip. Also, the force of the plunger tip may cause the IOL to twist and/or turn as it is moved through the inserter whereby the force between the IOL and the plunger tip and/or the inserter lumen may uncontrollably increase to the point of IOL damage.

Various inserter devices have been proposed which attempt to address these problems, and particularly the problem of controlling the force between the plunger, lumen and IOL, yet there remains a need for an IOL inserter which itself is simple in design and also simplifies operation of the IOL inserter device and thereby the IOL delivery process.

### SUMMARY OF THE INVENTION

The invention provides an inserter having more precise control of the plunger/inserter interface by providing an inserter with at least one, but preferably a pair of cantilevered projections integrally formed on the shaft of the plunger. The projections are biased radially outwardly of the plunger shaft and are designed to provide a biasing force against the interior wall of the inserter lumen. The inserter body may further include a respective pair of through holes or other features for interacting with the projections to prevent unintentional separation of the plunger from the inserter.

### BRIEF DESCRIPTION OF THE DRAWINGS

FIG. 1 is a perspective view of a prior art inserter device;

FIG. 2 is an enlarged, partial perspective view of the proximal end of the tubular body of the inventive inserter;

FIG. 2b is a cross-sectional view of the plunger and body as taken generally along the line 2b-2b in FIG. 2;

FIG. 3 is an enlarged, partial perspective view of the body component of the inventive inserter;

FIG. 4 is an enlarged, partial perspective view of the plunger component of the inventive inserter; and

FIG. 5 is an assembly view of FIGS. 3 and 4.

### DETAILED DESCRIPTION

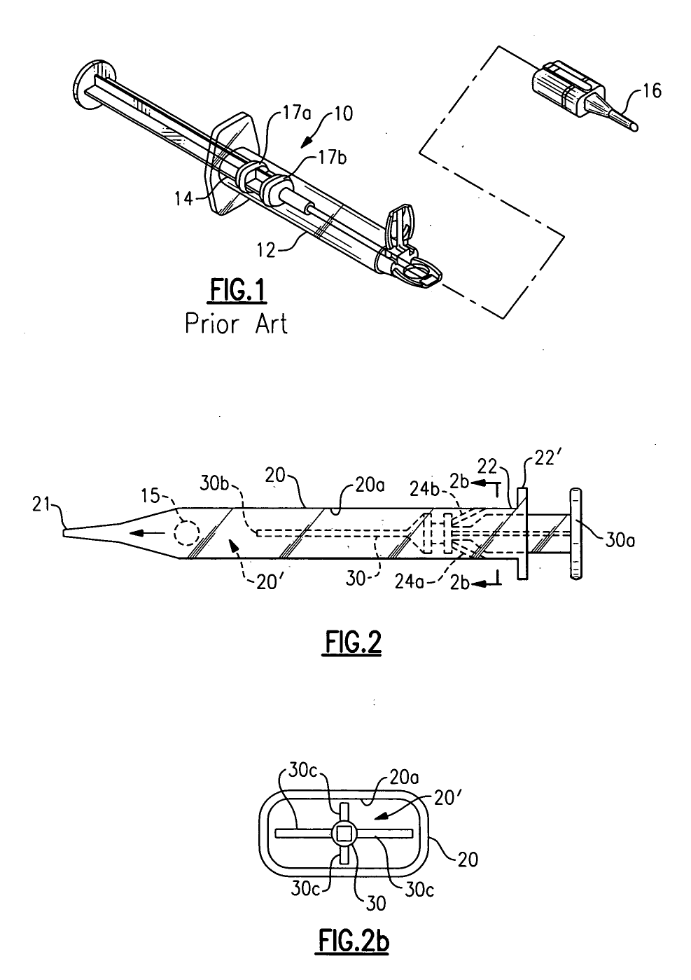

The present invention is directed towards IOL inserters having a tubular body defining a longitudinal passageway or lumen extending from a proximal end to a distal tip of the inserter, and a plunger component which telescopes in the open proximal end of the tubular body. A prior art inserter device 10 is seen in FIG. 1 to include a tubular body 12 having an open proximal end 14 and opposite distal tip 16 which is inserted into an incision in an eye for passing an IOL through the inserter, out tip 16 and into the eye. To control movement of the plunger within the body, the prior art device uses one or more rubber O-rings 17a, 17b which provide friction between the plunger and inside wall of the body. O-rings are separate pieces that require direct manual attachment to the plunger component thereby increasing the cost of manufacture. Furthermore, prior art device 10 does not include any feature to prevent unintentional retraction and/or disengagement of the plunger with respect to the inserter body. Even further, prior art device 10 does not include any feature to establish one or more predetermined advancement positions of the plunger with respect to the inserter body.

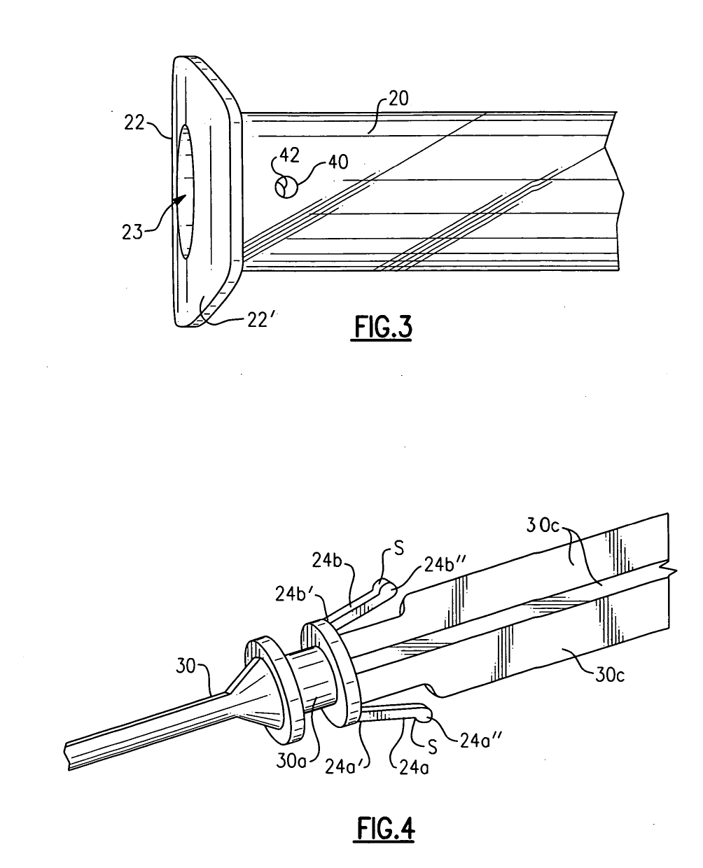

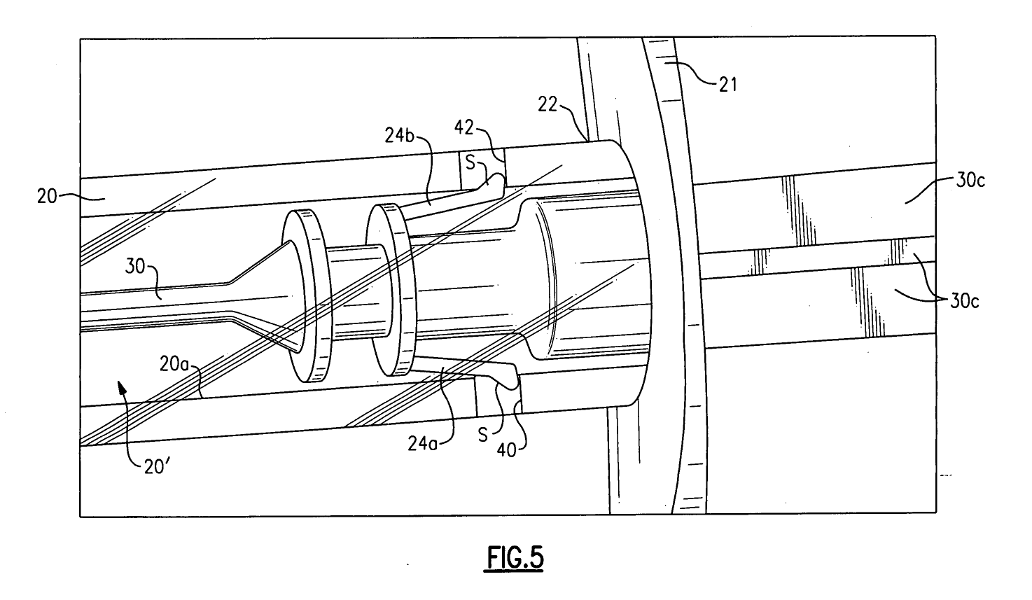

It is understood that the invention is directed at the plunger/body interface. The other parts of the inserter (e.g., the cross-sectional shape of the inserter body, the IOL loading area, the inserter distal tip, the plunger tip, etc.) may therefore be of any desired configuration. FIGS. 2-5 show a preferred embodiment of the inventive plunger/inserter body interface. The inserter body 20 has a distal tip 21 and a proximal end 22 having an opening 23 wherethrough the plunger component 30 telescopes. A simplified IOL 15 is seen loaded into body 20 through any known IOL loading means designed into the inserter body (not shown). Body proximal end 22 includes a finger hold 22′ and plunger 30 includes a finger press 30a at the proximal end thereof for pressing and advancing the plunger through the longitudinal passageway 20′ in the manner of a syringe. A plunger tip 30b is provided at the distal end thereof for engaging and pushing the IOL 15 through and out the distal tip 21 of the inserter body 20.

It is important that the plunger 30 be properly aligned and rotationally fixed within the passageway 20′ so as to ensure proper engagement of the IOL 15 with plunger tip 30b. Plunger alignment and rotational fixing can be achieved by forming the body 20 and proximal end of the plunger in a non-circular (e.g., oval) cross-sectional shape. The proximal end of the plunger may be provided with a plurality of radially extending fins 30c which act to align and rotationally fix plunger 30 within body passageway 20′ (see also FIG. 2b).

The frictional force between the plunger 30 and inside wall 20a of the body 20 is dictated by at least one, but preferably a pair of cantilevered projections 24a, 24b attached at first ends 24a′, 24b′ thereof to the plunger shaft 30a, respectively. The projections 24a, 24b are biased radially outward of the plunger shaft such that the free ends 24a″, 24b″ thereof are pressed inwardly as they telescope within passageway 20. As such, they exert a biasing force against the inside wall 20a of the inserter body thereby providing resistance to plunger movement within the inserter body. This resistive force is necessary to allow the surgeon to maintain precise control over plunger movement. The resistive force imparted by projections 24a, 24b is predetermined through design parameters which may include the material from which the parts are made, the dimensions of the projections, and the attachment angle of the projection relative to the plunger shaft. The desired frictional force and appropriate design parameters needed to achieve the desired force are determinable by those skilled in the art without undue experimentation.

As seen in FIGS. 2, 3 and 5, the inserter body 20 includes a like number of detents, e.g., recesses or through holes 40, 42, which are positioned to align with the free ends 24a′, 24b′ of projections 24a, 24b, respectively. As the plunger is advanced through passageway 20′, the free ends 24a′, 24b′ will encounter and become engaged within holes 40, 42 as seen in FIGS. 2 and 5. The shape of the free ends 24a′, 24b′ may be provided with a sloping surface “s” or other feature to prevent rearward movement (retraction) of the plunger once the free ends have become engaged in the holes. The inserter may therefore be packaged and presented to the user with the free ends 24a′, 24b′ thereof already engaged in their respective holes 40,42 to prevent unintentional separation of the plunger from the body during shipping or any time prior to use. At the same time, the sloping surface “s” should not impede continued advancement of the plunger (toward distal tip 21) upon application of a force larger than the retention force between the free ends and their associated holes. It will be appreciated that the free ends 24a′, 24b′ may be designed in a variety of configurations to achieve the desired resistive force.

It is furthermore noted that the placement of the holes, recesses or other detent features may be placed at any desired location along the inserter body 20. For example, if it is desired that the plunger tip be at a precise location at certain stages of inserter storage and/or use, the detent location may be chosen to fix the plunger tip at the desired longitudinal location within passageway 20′. Furthermore, more than one detent or set of detents may be positioned at more than one longitudinal location along body 20. For example, one detent set (such as holes 40,42) may be positioned close to proximal end 22 to fix the plunger during storage and shipping. Another detent set may be provided closer to distal end 21 to fix the plunger at the end of its advancement stroke. Another detent set may be provided near the middle of the body 20 to fix the plunger tip at a certain position from or even contacting the IOL 15. It is thus understood that the detents may be longitudinally spaced and strategically positioned along body 20 to achieve any desired incremental plunger advancement profiles.

1. An inserter for inserting an IOL into an eye, said inserter comprising:
a) an inserter body having a proximal end and a distal tip and an inside wall defining a longitudinal passageway extending therebetween;
b) a plunger having a proximal end and a distal plunger tip, said plunger telescoping within said longitudinal passageway of said body;
c) one or more cantilevered projections attached to and radially extending from said plunger, said projections configured to apply a biasing force against said inside wall of said inserter body.

2. The inserter of claim 1, and further comprising one or more detents formed in said injector body and configured for engaging with said one or more projections upon said projections encountering a respective said detent through movement of said plunger relative to said injector body, respectively.

3. The injector of claim 2 wherein said detents are through holes.

4. The injector of claim 2 and further comprising first and second projections on said plunger for engaging with first and second detents on said injector body, respectively.

5. The injector of claim 2 and further comprising a plurality of detents formed in longitudinally spaced relation along said injector body.

6. The injector of claim 1 wherein said projections have a respective free end which is configured to prevent retraction of said plunger with respect to said injector body when said projections are engaged in a respective detent.
